(19) United States
(12) Patent Application Publication
(10) Pub. No.: US 2023/0151363 A1
(43) Pub. Date: May 18, 2023

(54) MODIFIED SHORT-INTERFERING RNA COMPOSITIONS AND THEIR USE IN THE TREATMENT OF CANCER

(71) Applicant: THE RESEARCH FOUNDATION FOR THE STATE UNIVERSITY OF NEW YORK, Albany, NY (US)

(72) Inventors: Jingfang JU, East Setauket, NY (US); Andrew FESLER, Sound Beach, NY (US)

(73) Assignee: THE RESEARCH FOUNDATION FOR THE STATE UNIVERSITY OF NEW YORK, Albany, NY (US)

(21) Appl. No.: 17/912,688

(22) PCT Filed: Mar. 18, 2021
(86) PCT No.: PCT/US21/22867

§ 371 Date: Sep. 19, 2022

Related U.S. Application Data
(60) Provisional application No. 62/991,296, filed on Mar. 18, 2020.

(51) Int. Cl. C12N 15/113; A61P 35/00

(52) U.S. Cl. CPC C12N 15/113; A61P 35/00; C12N 2310/14; C12N 2310/335; C12N 2320/30

(57) ABSTRACT

The present disclosure provides modified short-interfering ribosomal nucleic acid compositions that have one or more uracil bases replaced a 5-fluorouracil molecule. More specifically, the present disclosure reveals that the replacement of uracil nucleotides within an siRNA nucleotide sequence with a 5-fluorouracil increases the ability of the short interfering RNA to inhibit cancer progression and tumorigenesis when compared to known cancer therapeutics. As such, the present disclosure provides various short-interfering nucleic acid compositions having 5-fluorouracil molecules incorporated in their nucleic acid sequences and methods for using the same. The present disclosure further provides pharmaceutical compositions comprising the modified nucleic acid compositions, and methods for treating cancers using the same.

### CROSS REFERENCE TO RELATED APPLICATIONS

This application claims the benefit of priority from U.S. Provisional Application No. 62/991,296, filed Mar. 18, 2020, the entire contents of which are incorporated herein by reference.

### GOVERNMENT SUPPORT

This invention was made with government support under grant number CA197098 awarded by the National Institutes of Health. The government has certain rights in the invention.

### INCORPORATION BY REFERENCE OF SEQUENCE LISTING

The Sequence Listing in the ASCII text file, named as 050_9019_US_Pro_SequenceListing.txt of 3 KB bytes, and submitted to the United States Patent and Trademark Office via EFS-Web, is incorporated herein by reference.

### FIELD OF THE DISCLOSURE

The present disclosure is generally directed to short-interfering ribosomal nucleic acid (siRNA) compositions that include 5-fluorouracil (5-FU) molecules. More specifically, the present disclosure provides modified siRNA compositions that contain one or more 5-FU molecules and methods for using the same. The instant application also provides pharmaceutical compositions that include the inventive short-interfering nucleic acid compositions and methods for treating cancer using the same.

### BACKGROUND

RNA interference (RNAi) refers to the process of sequence-specific post-transcriptional gene silencing in animals mediated by short-interfering RNAs. See e.g., Zamore et al., Cell (2000) 101:25-33 and Hamilton et al., Science (1999) 286:950-951. Briefly, the presence of double-stranded RNAs (dsRNAs) in cells stimulates the activity of a ribonuclease III enzyme referred to as dicer. See, e.g., Zamore et al., Cell. (2000) 101: 25-33. Dicer is involved in the processing of the dsRNA into short pieces of dsRNA known as short-interfering RNAs (siRNAs). Short-interfering RNAs derived from dicer activity are typically about 21 to about 23 nucleotides in length and comprise about 19 base pair duplexes. Id. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex. See Elbashir et al., Genes Dev., (2001) 15:188. RNAi has been studied extensively, for example, Tuschl et al., International PCT Publication No. WO 01/75164, describe RNAi induced by introduction of duplexes of synthetic 21-nucleotide RNAs in cultured mammalian cells including human embryonic kidney and HeLa cells.

RNA interference appears to be an effective technology to suppress target mRNA translation and the recent FDA approval of siRNA based therapy is a great demonstration of their therapeutic potential. Hoy, S M. Drugs (2018) 78: 1625-1631; and Schutze, N. Mol Cell Endocrinol (2004) 213: 115-119. However, over the years, siRNA-based therapy has been limited due to delivery vehicle toxicity and limited to certain organ sites such as liver. For example, these compounds are known to be susceptible to enzymatic degradation when administered, which results in poor stability. Nikam, R R and Gore, K R. Nucleic Acid Ther. (2018) 28: 209-224. In addition, studies concerning the use of siRNA in the art provide conflicting results. For example, studies have shown that complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence. See Elbashir et al., EMBO J. (2001) 20:6877. Other studies have indicated that a 5′-phosphate on the target-complementary strand of an siRNA duplex is required for siRNA activity and that ATP is utilized to maintain the 5′-phosphate moiety on the siRNA. See Nykanen et al., Cell (2001) 107:309. Furthermore, certain studies have shown that some base modifications, including substituting, in sense and antisense strands of the siRNA, 4-thiouracil, 5-bromouracil, 5-iodouracil, and 3-(aminoallyl) uracil for uracil, and inosine for guanosine revealed confusing and conflicting results. See Parrish et al., Molecular Cell (2000) 6:1077-1087. For example, 4-thiouracil and 5-bromouracil substitution appeared to be tolerated, while other substitutions such incorporation of 5-iodouracil and 3-(aminoallyl)uracil in the antisense strand resulted in a substantial decrease in RNAi activity. Id.

According to the World Health Organization, cancer is a leading cause of death worldwide, accounting for 8.8 million deaths in 2015. Lung cancer is the leading cause of cancer death in both men and women in the United States, with only 18.6% of patients diagnosed with lung cancer surviving beyond 5 years. Surveillance, Epidemiology, and End Results Program. SEER Cancer Stat Facts: Lung and Bronchus Cancer. National Cancer Institute. Bethesda, Md. (2018). There are two primary categories of lung cancer: non-small cell lung cancer and small cell lung cancer. Non-small cell lung cancer is further delineated by type of cancer cells present in a tissue. As such, non-small cell lung cancer is broken down into following sub-classes of lung cancer: squamous cell carcinoma (also called epidermoid carcinoma), large cell carcinoma, adenocarcinoma (i.e., cancer that originates in cells lining alveoli), pleomorphic, carcinoid tumor and salivary gland carcinoma. Meanwhile, there are two main types of small cell lung cancer: small cell carcinoma and combined small cell carcinoma. SEER Cancer Stat Facts: Lung and Bronchus Cancer. National Cancer Institute. Bethesda, Md. (2018). The most common treatment for non-small cell lung cancers is gemcitabine (2′, 2′-difluoro 2′deoxycytidine), taxol (e.g., paclitaxel), cisplatin (a DNA cross-linking agent), and combinations thereof. However, many types of antibody-based therapeutics are also used to treat non-small cell lung cancer (e.g., gefitinib, pembrolizumab, alectinib). Small cell lung cancer is commonly treated by methotrexate, doxorubicin hydrochloride, and topotecan based chemotherapeutic agents.

Colorectal cancer (CRC) is the third most common malignancy and the second most common cancer-related cause of death in the United States. See, Hegde S R, et al., Expert review of gastroenterology & hepatology. (2008) 2(1) pp. 135-49. There are many chemotherapeutic agents used to treat cancer; however pyrimidine antagonists, such as fluoropyrimidine-based chemotherapeutic agents (e.g., 5-fluorouracil, S-1) are the gold standard for treating colorectal cancer. Pyrimidine antagonists, block the synthesis of pyrimidine containing nucleotides (Cytosine and Thymine in DNA; Cytosine and Uracil in RNA). Because pyrimidine antagonists have similar structures when compared to endogenous nucleotides, they compete with the natural pyrimidines to inhibit crucial enzymatic activity involved in the replication process leading to the prevention of DNA and/or RNA synthesis and inhibition of cell division.

Lymphomas or cancers of the immune/lymphatic system, e.g., Hodgkin Lymphoma, Non-Hodgkin Lymphoma, are a common form of cancer. Generally, lymphomas include, for example, tumors of the lymph nodes, spleen, thymus gland and bone marrow. The primary types of lymphoma are Hodgkin lymphoma (i.e., Hodgkin's disease), non-Hodgkin's lymphoma, chronic lymphyocytic leukemia, cutaneous B-cell lymphoma, cutaneous T-cell lymphoma and Waldenstrom macroglobulinemia. Drugs approved for the treatment of lymphomas include, for example, doxorubicin hydrochloride, 5-FU, cyclophosphamide, dexamethasone, decarbazine, methotrexate, rituximab, ibrtinib, duvelisib, pembrolizumab, venetoclax and dasatinib.

5-fluorouracil (i.e., 5-FU, or more specifically, 5-fluoro-1H-pyrimidine-2,4-dione) is a well known pyrimidine antagonist that is used in many adjuvant chemotherapeutic medicants, such as Carac® cream, Efudex®, Fluoroplex®, and Adrucil®. It is well established that 5-FU targets a critical enzyme, thymidylate synthase (TYMS or TS), which catalyzes the methylation of deoxyuridine monophosphate (dUMP) to deoxythymidine monophosphate (dTMP) an essential step in DNA biosynthesis. Danenberg P. V., Biochim. Biophys. Acta. (1977) 473(2):73-92

Nevertheless, the existing cancer therapies are still in their infancy, with many hurdles still waiting to be improved or overcome. For example, it is well known that, although fairly efficacious in treating a variety of cancers, 5-FU possesses substantial toxicity and can elicit a host of adverse side effects. Moreover, tumor cells have been known to circumvent apoptotic pathways by developing resistance to common therapeutic agents, such as 5-FU. See Gottesman M. M. et al., Nature Reviews Cancer, (2002) 2(1):48-58.

B-cell lymphoma2 (Bcl-2) is a mitochondrial membrane protein encoded by the BCL2 gene, and is the founding member of the Bcl-2 family of regulator proteins that inhibit programmed cell death (apoptosis). Cory, S and Adams, J M T. Nat Rev Cancer (2002). 2: 647-656. Therefore, many attempts have been made to target BCL2 as a therapeutic strategy to combat cancer including FDA approved Venetoclax. See Leverson, J D et al. Cancer Discov (2017) 7: 1376-1393.

In view of the foregoing, there would be a significant benefit in more efficacious, stable, and less toxic medications for the treatment of cancer.

### SUMMARY OF THE DISCLOSURE

Without being bound by any one particular theory, the present disclosure is premised on the discovery that replacing uracil (U) bases within the nucleotide sequence of short-interfering RNA (siRNA) molecules with 5-FU molecules increases efficacy of 5-FU by providing 5-FU to a cell where the siRNA will target BCL-2, inhibit BCL-2 protein synthesis by binding a BCL-2 nucleotide sequence (mRNA), and releasing 5-FU intracellularly to inhibit thymidylate synthase (TS) to treat cancer, such as colorectal cancer, lung cancer and lymphoma.

The current disclosure demonstrates that modified siRNA, which replace at least one uracil base with a 5-FU molecule have exceptional efficacy as anti-cancer agents. Moreover, the data herein shows that contacting a cell with a modified siRNA composition of the present disclosure treats cancer by inhibiting cancer cell proliferation through modulating the apoptotic pathway. Furthermore, it is shown that the modified siRNAs of the present disclosure retain BCL-2 nucleic acid sequence target specificity, can be delivered without the use of harmful and ineffective delivery vehicles (e.g., nanoparticles), and exhibit enhanced potency when compared to known BCL-2 therapeutic agents (e.g., Venetoclax).

Therefore, in one aspect of the present disclosure, nucleic acid compositions that include a modified siRNA nucleotide sequence having at least one uracil base (U, U-bases) that has been replaced by a 5-FU molecule are described. In certain embodiments, the modified siRNA has more than one, or exactly one uracil that has been replaced by 5-flurouracil. In some embodiments, the modified siRNA nucleotide sequence replaces two, three, four, five or more uracil bases with a 5-FU molecule. In specific embodiments, all of the uracil bases of an anti BCL-2 short-interfering RNA have each been replaced by a 5-FU molecule.

In other embodiments, one or more of the uracil bases in a modified siRNA composition have been replaced in a first strand of a double-stranded siRNA molecule. In another embodiment, all of the uracil bases in a first strand of a double-stranded anti-BCL-2 short-interfering RNA have each been replaced by a 5-FU molecule. In a certain embodiments, one or more of the uracil bases in a modified siRNA composition have been replaced in a first strand of a double-stranded siRNA molecule and one or more of the uracil bases have been replaced in the second strand of the double-stranded siRNA molecule. In other embodiments, one or more of the uracil bases in a modified siRNA composition have been replaced in a first strand of a double-stranded siRNA molecule and none of the uracil bases have been replaced by a 5-FU molecule in the second strand of the double stranded siRNA molecule. In a specific embodiment, all of the uracil bases in a modified siRNA composition have been replaced by a 5-FU molecule in a first strand of a double-stranded siRNA molecule and one or more of the uracil bases have been replaced in the second strand of the double-stranded siRNA molecule. In one embodiment, all of the uracil bases in a modified siRNA composition have been replaced by a 5-FU molecule in a first strand of a double-stranded siRNA molecule and none of the uracil bases have been replaced in the second strand of the double-stranded siRNA molecule. In some embodiments, the first strand is the sense strand of the double-stranded siRNA molecule. In other embodiments, the first strand is the sense strand of the double-stranded siRNA molecule and the second strand is the antisense strand.

In a specific embodiment, the nucleic acid composition includes a double-stranded siRNA nucleotide sequence that has been modified by replacing at least one of the uracil bases with a 5-FU molecule. More specifically, the nucleic acid composition is a double-stranded RNA molecule that contains at least the following nucleotide sequence, from 5′ to 3′, which binds to a portion of the BCL-2 mRNA nucleotide sequence: GGAUGCCUUUGUGGAACUGUAUU [SEQ ID NO. 1] and the complementary strand, wherein at least one, two, three, four, five, six, seven or all of the uracil bases are replaced by a 5-FU molecule.

In one instance, a modified siRNA of the present disclosure includes precisely one uracil base of the siRNA nucleotide sequence that has been replaced by a 5-FU molecule. In other instances, precisely or at least two uracil bases in the siRNA nucleotide sequence are each replaced by a 5-FU molecule. In yet other instances, precisely or at least three uracil bases in the siRNA nucleotide sequence are each replaced by a 5-FU molecule. In another instance, precisely or at least four uracil bases in the siRNA nucleotide sequence are each replaced by a 5-FU molecule. In another instance, precisely or at least five uracil bases in the siRNA nucleotide sequence are each replaced by a 5-FU molecule. In other embodiments, precisely or at least six uracil bases in the siRNA nucleotide sequence are each replaced by a 5-FU molecule. In another embodiment, precisely or at least seven uracil bases in the siRNA nucleotide sequence are each replaced by a 5-FU molecule. In specific embodiments, all of the uracil bases of the siRNA nucleotide sequence are each replaced by a 5-FU molecule. The modifications to any siRNA composition of the present disclosure can be made to a first strand (e.g., sense strand) or the complementary second strand (e.g., antisense strand) of the double-stranded siRNA composition. In a preferred embodiment, the modifications to the siRNA molecule are made to both the first (sense) strand and second (antisense) strand.

In an exemplary embodiment, the nucleic acid composition of the present disclosure has a modified siRNA nucleotide sequence of, from 5′ to 3′, which binds to BCL-2 mRNA: GGAU^{F}GCCU^{F}U^{F}U^{F}GU^{F}GGAACU^{F}GU^{F}AU^{F}U^{F}, wherein Ur is a 5-FU molecule and a complementary antisense strand (from 3′ to 5′) wherein each uracil base is replaced by a 5-FU molecule as set forth in SEQ ID NO. 2.

In an another embodiment, the nucleic acid composition of the present disclosure has a modified siRNA nucleotide sequence of, from 3′ to 5′, which binds to BCL-2 mRNA: UUCCUACGGAAACACCUUGACAU and a complementary sense strand wherein each uracil base is replaced by a 5-FU molecule as set forth in SEQ ID NO: 3.

In yet another embodiment, the nucleic acid composition of the present disclosure has a modified siRNA nucleotide sequence of, from 3′ to 5′, which binds to BCL-2 mRNA: U^{F}U^{F}CCU^{F}ACGGAAACACCU^{F}U^{F}GACAU^{F} and a complementary sense strand wherein none of the uracil bases are replaced by a 5-FU molecule as set forth in SEQ ID NO: 4.

The present disclosure also contemplates modified siRNA compositions with at least one uracil base replaced by a 5-halouracil other than 5-fluorouracil. Therefore, in some modified siRNA compositions of the present disclosure one or more uracil base is replaced by for example, 5-chlorouracil, 5-bromouracil, 5-iodouracil, 5-flurouracil or a combination thereof. In certain embodiments, the modified siRNA nucleotide sequence includes more than one 5-halouracil whereby each of the 5-halouracils are the same. In other embodiments, the modified siRNA nucleotide sequence includes more than one 5-halouracil whereby each of the 5-halouracils is different. In other embodiments, the modified siRNA nucleotide sequence includes more than two 5-halouracils, whereby the modified siRNA nucleotide sequence includes a combination of different 5-halouracils.

The present disclosure is also directed to formulations containing the modified siRNA compositions described herein or a formulation that includes combinations thereof, i.e., at least two different modified siRNAs. In certain embodiments, the formulations can include pharmaceutical preparations that comprise the above-described nucleic acid compositions and other known pharmacological agents, such as one or more pharmaceutically acceptable carriers.

The present disclosure reveals that the inventive modified siRNAs exhibit a potent efficacy as an anti-cancer therapeutic. Notably, each of the modified siRNA nucleic acid compositions tested reduce cancer cell viability, tumor growth and development.

Therefore, another aspect of the present disclosure is directed to a method for treating cancer that includes administering to a subject an effective amount of one or more of nucleic acid compositions described herein. In certain embodiments of the present methods, the nucleic acid compositions include a modified siRNA that binds to BCL-2 mRNA, wherein at least one, two, three, four, five, six, seven or more of the uracil bases are replaced by a 5-fluorouracil molecule.

In a specific embodiment, the siRNA composition of the present disclosure binds to BCL-2 mRNA and has a modified nucleotide sequence of, from 5′ to 3′: GGAU^{F}GCCU^{F}U^{F}U^{F}GU^{F}GGAACU^{F}GU^{F}AU^{F}U^{F}, wherein U^{F} is a 5-FU molecule and a complementary antisense strand (from 3′ to 5′) wherein each uracil base is replaced by a 5-FU molecule as set forth in SEQ ID NO. 2.

In an another embodiment, the nucleic acid composition of the present disclosure binds to BCL-2 mRNA and has a modified siRNA nucleotide sequence of, from 3′ to 5′: UUCCUACGGAAACACCUUGACAU and a complementary sense strand wherein each uracil base is replaced by a 5-FU molecule as set forth in SEQ ID NO: 3.

In yet another embodiment, In an another embodiment, the nucleic acid composition of the present disclosure binds to BCL-2 mRNA and has a modified siRNA nucleotide sequence of, from 3′ to 5′, which binds to BCL-2 mRNA: U^{F}U^{F}CCU^{F}ACGGAAACACCU^{F}U^{F}GACAU^{F} and a complementary sense strand wherein none of the uracil bases are replaced by a 5-FU molecule as set forth in SEQ ID NO: 4.

In some instances, the subject being treated by the present methods is a mammal. In certain embodiments, the subject being treated is a human, dog, horse, pig, mouse, or rat. In a specific embodiment, the subject is a human that has been diagnosed with cancer, or has been identified as having a predisposition to developing cancer. In some embodiments, the cancer being treated can be, for example, lung cancer, colorectal cancer or lymphoma. In a specific embodiment, the cancer being treated is colorectal cancer. In certain embodiments, the cancer being treated is lung cancer. In one embodiment, the cancer being treated is lymphoma.

### BRIEF DESCRIPTION OF THE DRAWINGS

FIGS. 1A-1D. Chemical representation of exemplary short-interfering RNA nucleotide sequences of the present disclosure. A) Chemical representation of an unmodified short-interfering BCL-2 RNA set forth in SEQ ID NO: 1 (siBCL2). B) Chemical representation of an exemplary modified siBCL2 RNA whereby all uracil residues in both the sense and antisense strand were replaced in siBCL2 by 5-FU as set forth in SEQ ID NO: 2. C) Chemical representation of an exemplary modified siBCL2 RNA whereby all uracil residues in the sense strand were replaced in siBCL2 by 5-FU as set forth in SEQ ID NO: 3. D) Chemical representation of an exemplary modified siBCL2 RNA whereby all uracil residues in the antisense strand were replaced in siBCL2 by 5-FU as set forth in SEQ ID NO: 4. The orientation of each siRNA depicted is provided by a 5′ to 3′ (sense) or 3′ to 5′ (antisense) designation.

FIGS. 2A-2C. An exemplary modified siRNA molecule maintains BCL2 target specificity and the ability to inhibit target (BCL-2) expression. A) qRT-PCR analysis shows that in colon cancer cells (HCT 116) and lung cancer cells (A549), the exemplary modified siRNA of SEQ ID NO: 2 (5-FU-siBCL2) inhibits BCL-2 at the mRNA level. (P<0.001) B) 5-FU-siBCL2 inhibits BCL-2 expression, and thus cancer progression with or without a transfection vehicle. Western blot demonstrates in HCT 116 colon cancer cells that the exemplary modified siRNA of SEQ ID NO: 2 (5-FU-siBCL2) inhibits BCL-2 expression at the protein level with or without a transfection vehicle and this is not the effect of 5-FU alone. C) Western blot demonstrates in A549 lung cancer cells that the exemplary modified siRNA of SEQ ID NO: 2 (5-FU-siBCL2) inhibits BCL-2 expression at the protein level with or without a transfection vehicle and this is not the effect of 5-FU alone.

FIGS. 3A-3D. An exemplary modified siRNA molecule induces apoptosis in colon cancer and lymphoma cells and is more effective in killing cancer cells than known therapeutic agents. A) With or without transfection vehicle, 50 nM of the exemplary modified siRNA of SEQ ID NO: 2 (5-FU-siBCL2) induces apoptosis in HCT 116 colon cancer cells. (P<0.05) B) With or without transfection vehicle, 50 nM of the exemplary modified siRNA of SEQ ID NO: 2 (5-FU-siBCL2) induces apoptosis in Toledo lymphoma cells. (P<0.05)C) 5-FU-siBCL2 is more effective at inducing apoptosis than Venetoclax. (P<0.05) D) 5-FU-siBCL2 inhibits lymphoma cell viability at a lower dose than Venetoclax.

### DETAILED DESCRIPTION OF THE DISCLOSURE

The present disclosure provides short-interfering ribosomal nucleic acid (siRNA) compositions that bind to a BCL-2 nucleic acid sequence, and incorporates one or more 5-fluorouracil (5-FU) molecules. Without being bound by any one particular theory, surprisingly, the present disclosure reveals that the replacement of uracil nucleotides within at least one strand of a double-stranded siRNA composition that binds to BCL-2 mRNA with a 5-halouracil (e.g., 5-fluorouracil) increases the ability of the siRNA to inhibit cancer development, progression and tumorigenesis. Moreover, the data herein shows that contacting a several types of cancer cells with a modified siRNA compositions of the present disclosure reduces cancer progression by modulating the apoptotic pathway through the suppression of BCL-2 mRNA translation. Furthermore, it is shown that the inventive modified siRNAs retain target specificity to BCL-2 mRNA, can be delivered without the use of harmful and ineffective delivery vehicles (e.g., nanoparticles), and exhibit enhanced potency when compared to unmodified siRNA compositions that bind to BCL-2 mRNA. As such, the present disclosure provides various short-interfering nucleic acid compositions having 5-fluorouracil molecules incorporated in their nucleic acid sequences and methods for using the same to treat cancer. The present disclosure further provides pharmaceutical formulations composed of the modified siRNA compositions, and methods for treating cancers that include administration of the same to a subject in need thereof.

### Modified Short-Interfering Ribosomal Nucleic Acid Compostions

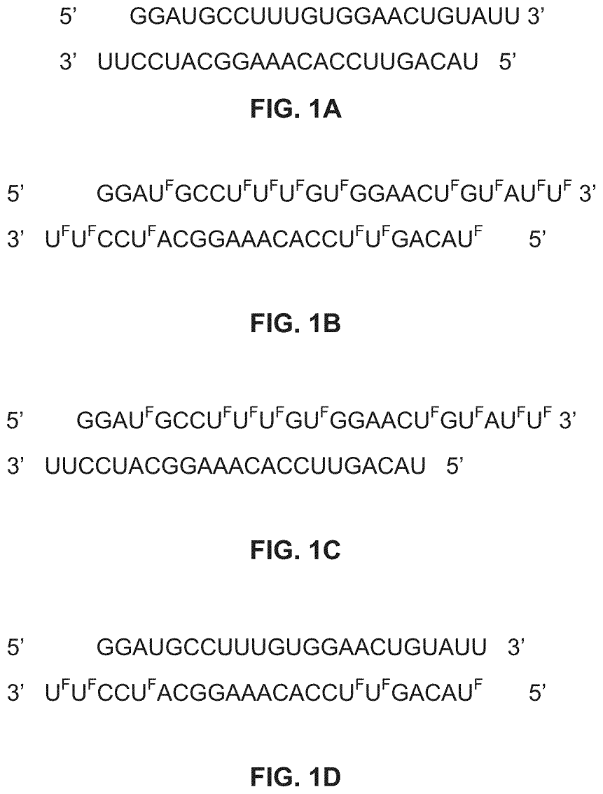

The term “short-interfering RNA”, “siRNA molecule” and “siRNA” are used interchangeably herein to mean any nucleic acid molecule capable of inhibiting or down regulating gene expression or viral replication, for example by mediating RNA interference “RNAi” or gene silencing in a nucleotide sequence-specific manner. Non limiting examples of siRNA molecules of the invention are shown in FIGS. 1B-ID, and Examples 1-2 herein. For example the siRNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence (e.g., BCL-2) or a portion thereof. The siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand); such as where the antisense strand and sense strand form a duplex or double-stranded structure, for example wherein the double-stranded region is about 15 to about 30, e.g., 14, 15, 16, 17, 18, 19, 20, 21, 22, 23, 24, 25, 26, 27, 28, 29, 30 or 31 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., 15 to 25 or more nucleotides of the siRNA molecule are complementary to the target nucleic acid or a portion thereof). In certain embodiments, the term siRNA incorporates both the duplex (i.e., double-stranded) form of the siRNA, and single-stranded form of the siRNA in either the 5′ to 3′ direction and complementary strand in the 3′ to 5′ direction. In specific embodiments, modified siRNA compositions of the present disclosure are composed of a double-stranded composition having a first strand and a second strand that are complementary to each other.

The term “complementarity” or “complementary” as used herein shall mean that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art. See, e.g., Frier et al., Proc. Nat. Acad. Sci. USA (1986) 83:9373-9377. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence. In one embodiment, an siRNA molecule of the invention comprises about 15 to about 30 or more (e.g., 14, 15, 16, 17, 18, 19, 20, 21, 22, 23, 24, 25, 26, 27, 28, 29, 30, 31 or more) nucleotides that are complementary to one or more corresponding nucleic acid molecule or a portion thereof.

The term “modified siRNA” and “modified short-interfering RNA” are used interchangeably herein to refer to a siRNA molecule that includes at least one 5-halouracil molecule. More specifically, in the present disclosure a modified siRNA differs from the unaltered or unmodified siRNA nucleic acid sequence by one or more base. In some embodiments of the present disclosure, a modified siRNA of the present disclosure includes at least one uracil (U) nucleotide base replaced by a 5-halouracil. In some embodiments, the nucleic acid compositions contain a nucleotide sequence that has been modified by derivatizing at least one of the uracil nucleobases at the 5-position with a group that provides a similar effect as a halogen atom. In some embodiments, the group providing the similar effect has a similar size in weight or spatial dimension to a halogen atom, e.g., a molecular weight of up to or less than 20, 30, 40, 50, 60, 70, 80, 90, or 80 g/mol. In certain embodiments, the group providing a similar effect as a halogen atom may be, for example, a methyl group, trihalomethyl (e.g., trifluoromethyl) group, pseudohalide (e.g., trifluoromethanesulfonate, cyano, or cyanate) or deuterium (D) atom. The group providing a similar effect as a halogen atom may be present in the absence of or in addition to a 5-halouracil base in the siRNA nucleotide sequence.

In a specific embodiment, a modified siRNA of the present disclosure includes at least one uracil (U) nucleotide base replaced by a 5-fluorouracil.

The present disclosure also contemplates modified siRNA compositions with at least one uracil base replaced by a 5-halouracil other than 5-fluorouracil. Therefore, in some modified siRNA compositions of the present disclosure one or more uracil base is replaced by for example, 5-chlorouracil, 5-bromouracil, 5-iodouracil, 5-flurouracil or a combination thereof. In certain embodiments, the modified siRNA nucleotide sequence includes more than one 5-halouracil whereby each of the 5-halouracils are the same. In other embodiments, the modified siRNA nucleotide sequence includes more than one 5-halouracil whereby each of the 5-halouracils is different. In other embodiments, the modified siRNA nucleotide sequence includes more than two 5-halouracils, whereby the modified siRNA nucleotide sequence includes a combination of different 5-halouracils.

In certain embodiments, the modified siRNA nucleotide sequence includes more than one 5-halouracil whereby each of the 5-halouracils are the same. In other embodiments, the modified siRNA nucleotide sequence includes more than one 5-halouracil whereby each of the 5-halouracils is different. In other embodiments, the modified siRNA nucleotide sequence includes more than two 5-halouracils, whereby the modified siRNA nucleotide sequence includes a combination of different 5-halouracils.

In an exemplary embodiment of the present disclosure, a nucleic acid composition that contains a siRNA nucleotide sequence set forth in SEQ ID NO:1 that has been modified by replacing at least one of the uracil nucleotide bases with a 5-halouracil such as 5-fluorouracil is provided. In certain embodiments, the modified siRNA has more than one, or exactly one uracil that has been replaced by 5-flurouracil. In some embodiments, the modified siRNA nucleotide sequence replaces two, three, four, five or more uracil bases with a 5-FU molecule. In specific embodiments, all of the uracil bases of an anti BCL-2 short-interfering RNA have each been replaced by a 5-FU molecule.

In other embodiments, one or more of the uracil bases in a modified siRNA composition have been replaced in a first strand of a double-stranded siRNA molecule. In another embodiment, all of the uracil bases in a first strand of a double-stranded anti BCL2 short-interfering RNA have each been replaced by a 5-FU molecule. In a certain embodiments, one or more of the uracil bases in a modified siRNA composition have been replaced in a first strand of a double-stranded siRNA molecule and one or more of the uracil bases have been replaced in the second strand of the double-stranded siRNA molecule. In other embodiments, one or more of the uracil bases in a modified siRNA composition have been replaced in a first strand of a double-stranded siRNA molecule and none of the uracil bases have been replaced by a 5-FU molecule in the second strand of the double-stranded siRNA molecule. In a specific embodiment, all of the uracil bases in a modified siRNA composition have been replaced by a 5-FU molecule in a first strand of a double-stranded siRNA molecule and one or more of the uracil bases have been replaced in the second strand of the double-stranded siRNA molecule. In one embodiment, all of the uracil bases in a modified siRNA composition have been replaced by a 5-FU molecule in a first strand of a double-stranded siRNA molecule and none of the uracil bases have been replaced in the second strand of the double-stranded siRNA molecule. In some embodiments, the first strand is the sense strand of the double-stranded siRNA molecule. In other embodiments, the first strand is the sense strand of the double-stranded siRNA molecule and the second strand is the antisense strand.

In a specific embodiment, the nucleic acid composition includes a double stranded siRNA nucleotide sequence that has been modified by replacing at least one of the uracil bases with a 5-FU molecule. More specifically, the nucleic acid composition is a double stranded RNA molecule that contains at least the following nucleotide sequence, from 5′ to 3′, which binds to a portion of a BCL-2 nucleotide sequence: GGAUGCCUUUGUGGAACUGUAUU [SEQ ID NO. 1] and the complementary strand, wherein at least one, two, three, four, five, six, seven or all of the uracil bases are replaced by a 5-FU molecule.

In one instance, a modified siRNA of the present disclosure includes precisely one uracil base of the siRNA nucleotide sequence that has been replaced by a 5-FU molecule. In other instances, precisely or at least two uracil bases in the siRNA nucleotide sequence are each replaced by a 5-FU molecule. In yet other instances, precisely or at least three uracil bases in the siRNA nucleotide sequence are each replaced by a 5-FU molecule. In another instance, precisely or at least four uracil bases in the siRNA nucleotide sequence are each replaced by a 5-FU molecule. In another instance, precisely or at least five uracil bases in the siRNA nucleotide sequence are each replaced by a 5-FU molecule. In other embodiments, precisely or at least six uracil bases in the siRNA nucleotide sequence are each replaced by a 5-FU molecule. In another embodiment, precisely or at least seven uracil bases in the siRNA nucleotide sequence are each replaced by a 5-FU molecule. In specific embodiments, all of the uracil bases of the siRNA nucleotide sequence are each replaced by a 5-FU molecule. The modifications to any siRNA composition of the present disclosure can be made to a first strand (e.g., sense strand) or the complementary second strand (e.g., antisense strand) of the double-stranded siRNA composition. In a preferred embodiment, the modifications to the siRNA molecule are made to both the first (sense) strand and second (antisense) strand.

In an exemplary embodiment, the nucleic acid composition of the present disclosure has a modified siRNA nucleotide sequence of, from 5′ to 3′, which binds to BCL-2 mRNA: GGAU^{F}GCCU^{F}U^{F}U^{F}GU^{F}GGAACU^{F}GU^{F}AU^{F}U^{F}, wherein Ur is a 5-FU molecule and a complementary antisense strand (from 3′ to 5′) wherein each uracil base is replaced by a 5-FU molecule as set forth in SEQ ID NO. 2.

In an another embodiment, the nucleic acid composition of the present disclosure has a modified siRNA nucleotide sequence of, from 3′ to 5′, which binds to BCL-2 mRNA: UUCCUACGGAAACACCUUGACAU and a complementary sense strand wherein each uracil base is replaced by a 5-FU molecule as set forth in SEQ ID NO: 3.

In yet another embodiment, the nucleic acid composition of the present disclosure has a modified siRNA nucleotide sequence of, from 3′ to 5′, which binds to BCL-2 mRNA: U^{F}U^{F}CCU^{F} ACGGAAACACCU^{F}U^{F}GACAU^{F} and a complementary sense strand wherein none of the uracil bases are replaced by a 5-FU molecule as set forth in SEQ ID NO: 4.

The modified siRNA nucleic acid compositions described herein can be synthesized using any of the well known methods for synthesizing nucleic acids. In particular embodiments, the nucleic acid compositions are produced by automated oligonucleotide synthesis, such as any of the well-known processes using phosphoramidite chemistry. To introduce one or more 5-halouracil molecules such as a 5-FU into a modified siRNA nucleotide sequence, a 5-halouracil nucleoside phosphoramidite can be included as a precursor base, along with the phosphoramidite derivatives of nucleosides containing natural bases (e.g., A, U, G, and C) to be included in the nucleic acid sequence.

In some embodiments, the nucleic acid compositions of the present disclosure may be produced biosynthetically, such as by using in vitro RNA transcription from plasmid, PCR fragment, or synthetic DNA templates, or by using recombinant (in vivo) RNA expression methods, such as for example as in 2′-ACE RNA synthesis” as set forth, for example in S. A. Scaringe, et al., J. Am. Chem. Soc., (1998) 120 pp. 11820-11821, the entire contents of which is hereby incorporated by reference. See also C. M. Dunham et al., Nature Methods, (2007) 4(7), pp. 547-548.

The modified siRNA sequences of the present disclosure may be further chemically modified such as by functionalizing with polyethylene glycol (PEG) or a hydrocarbon or a targeting agent, particularly a cancer cell targeting agent, such as folate, by techniques well known in the art. To include such groups, a reactive group (e.g., amino, aldehyde, thiol, or carboxylate group) that can be used to append a desired functional group may first be included in the oligonucleotide sequence. Although such reactive or functional groups may be incorporated onto the as-produced nucleic acid sequence, reactive or functional groups can be more facilely included by using an automated oligonucleotide synthesis in which non-nucleoside phosphoramidites containing reactive groups or reactive precursor groups are included.

In certain embodiments, the modified siRNA compositions of the present disclosure are duplex molecules generated by synthesizing a first (oligonucleotide sequence) strand of the siRNA molecule, wherein the nucleotide sequence of the first strand comprises a cleavable linker molecule that can be used as a scaffold for the synthesis of the second strand; synthesizing the nucleotide sequence of the second strand of si RNA on the scaffold of the first strand, wherein the second strand sequence further comprises a chemical moiety than can be used to purify the siRNA duplex; cleaving the linker molecule under conditions suitable for the two siRNA strands to hybridize and form a stable duplex; and purifying the siRNA duplex utilizing the chemical moiety of the second oligonucleotide sequence strand.

In some embodiments, cleavage of the linker molecule above takes place during deprotection of the oligonucleotide, for example, under hydrolysis conditions using an alkylamine base such as methylamine. In one embodiment, the method of synthesis comprises solid phase synthesis on a solid support such as controlled pore glass (CPG) or polystyrene, wherein the first strand is synthesized on a cleavable linker, such as a succinyl linker, using the solid support as a scaffold. The cleavable linker can be used as a scaffold for synthesizing the second strand can comprise similar reactivity as the solid support derivatized linker, such that cleavage of the solid support derivatized linker and the cleavable linker takes place concomitantly. In another embodiment, the chemical moiety of that can be used to isolate the attached oligonucleotide sequence comprises a trityl group, for example a dimethoxytrityl group, which can be employed in a trityl-on synthesis strategy as described herein. In yet another embodiment, the chemical moiety, such as a dimethoxytrityl group, is removed during purification, for example, using acidic conditions.

In another instance, the method for siRNA synthesis is a solution phase synthesis or hybrid phase synthesis wherein both strands of the siRNA duplex are synthesized in tandem using a cleavable linker attached to the first sequence which acts a scaffold for synthesis of the second sequence. Cleavage of the linker under conditions suitable for hybridization of the separate siRNA strands results in formation of the double-stranded siRNA molecule.

In certain instances, the modified siRNA compositions of the present disclosure modulate BCL-2 protein expression.

The term “modulate” is meant that the expression of a gene, or level of RNA molecule or equivalent RNA molecules encoding one or more proteins or protein subunits of a gene, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit”, “down-regulate”, or “reduce”, it is meant that the expression of a gene, or level of mRNA molecules or equivalent RNA molecules encoding one or more proteins or protein subunits, or activity of one or more proteins or protein subunits, is reduced below that observed in the absence of the nucleic acid molecules (e.g., siRNA) of the invention. In one embodiment, inhibition, down-regulation or reduction with a modified siRNA molecule is below that level observed in the presence of an inactive or control molecule or in the absence of an siRNA molecule. In another embodiment, inhibition, down-regulation, or reduction with siRNA molecules is below that level observed in the presence of, for example, an siRNA molecule with scrambled sequence or with mismatches. In another embodiment, inhibition, down-regulation, or reduction of expression with a modified siRNA composition of the instant invention is greater in the presence of the modified siRNA molecule than in its absence or in the presence of an unmodified siRNA molecule. In one embodiment, inhibition, down regulation, or reduction of expression is associated with post transcriptional silencing, such as RNAi mediated cleavage of a target nucleic acid molecule (e.g. RNA or mRNA) or inhibition of translation of a gene product. In one embodiment, inhibition, down regulation, or reduction expression is associated with pretranslational silencing of BCL-2 mRNA in a cell.

The term “B-cell lymphoma 2” or “BCL-2” or “BCL2” means the gene, RNA transcript and protein set forth in RefSeq NG_009361.1, NM_000633, NP_000624, respectively, including portions thereof and isoform α (NM_000633.2, NP_000624.2) and R NM_000657.2, NP_000648.2 thereof which are encoded by the Bcl-2 gene, which is a member of the BCL-2 family of regulator proteins that regulate mitochondria regulated cell death via the intrinsic apoptosis pathway. BCL-2 is well known integral outer mitochondrial membrane protein that blocks the apoptotic death of cell cells by binding BAD and BAK proteins. For example, there are many known BCL-2 inhibitors such as Non-limiting examples of BCL2 inhibitors include Venetoclax (C₄₅H₅₀ClN₇O₇S, Genentech, Inc.), antisense oligonucleotides, such as Oblimersen (Genasense: Genta Inc.,), BH3 mimetic small molecule inhibitors including, ABT-737 (Abbott Laboratories, Inc.), ABT-199 (Abbott Laboratories, Inc.), and Obatoclax (Cephalon Inc.).

### Modified Short-Interfering Ribosomal Nucleic Acid Formulations

The present disclosure reveals that the inventive modified siRNA composition exhibit a potent efficacy as an anti-cancer therapeutic. As such, the present disclosure is also directed to formulations that include the modified siRNA compositions described herein or a formulation that includes combinations thereof, i.e., at least two different modified siRNAs. In certain embodiments, the formulations can include pharmaceutical preparations that comprise the above-described nucleic acid compositions and other known pharmacological agents, such as one or more pharmaceutically acceptable carriers.

In a some embodiments, the formulation is composed of an siRNA composition of the present disclosure that binds to a BCL-2 nucleotide sequence. In a specific embodiment, the formulation comprises a short-interfering RNA composition that has a modified nucleotide sequence that binds to BCL-2 mRNA.

In certain instances, the formulation comprises a short-interfering RNA composition that has a modified nucleotide sequence of, from 5′ to 3′: GGAU^{F}GCCUFU^{F}U^{F}GU^{F}GGAACU^{F}GU^{F}AU^{F}U^{F}, wherein Ur is a 5-FU molecule and a complementary antisense strand (from 3′ to 5′) wherein each uracil base is replaced by a 5-FU molecule as set forth in SEQ ID NO. 2. In an another embodiment, the formulation comprises a short-interfering RNA composition that has a modified nucleotide sequence that binds to BCL-2 mRNA and has a modified siRNA nucleotide sequence of, from 3′ to 5′: UUCCUACGGAAACACCUUGACAU and a complementary sense strand wherein each uracil base is replaced by a 5-FU molecule as set forth in SEQ ID NO: 3. In yet another embodiment, the formulation comprises a short-interfering RNA composition that has a modified nucleotide sequence that binds to BCL-2 mRNA and has a modified siRNA nucleotide sequence of, from 3′ to 5′, which binds to BCL-2 mRNA: U^{F}U^{F}CCU^{F}ACGGAAACACCU^{F}U^{F}GACAU^{F} and a complementary sense strand wherein none of the uracil bases are replaced by a 5-FU molecule as set forth in SEQ ID NO: 4.

The term “pharmaceutically acceptable carrier” is used herein as synonymous with a pharmaceutically acceptable diluent, vehicle, or excipient. Depending on the type of formulation or siRNA composition therein and intended the mode of administration, the siRNA composition may be dissolved or suspended (e.g., as an emulsion) in the pharmaceutically acceptable carrier. The pharmaceutically acceptable carrier can be any of those liquid or solid compounds, materials, compositions, and/or dosage forms which are, within the scope of sound medical judgment, suitable for use in contact with tissues of a subject. The carrier should be “acceptable” in the sense of being not injurious to the subject it is being provided to and is compatible with the other ingredients of the formulation, i.e., does not alter their biological or chemical function.

Some, non-limiting examples, of materials which can serve as pharmaceutically acceptable carriers include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. The pharmaceutically acceptable carrier may also include a manufacturing aid (e.g., lubricant, talc magnesium, calcium or zinc stearate, or stearic acid), a solvent, or encapsulating material. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19^{th} Ed. Mack Publishing Company, Easton, Pa., (1995).

In some embodiments, the pharmaceutically acceptable carrier may include diluents that increase the bulk of a solid pharmaceutical composition and make the pharmaceutical dosage form easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

In certain embodiments, a formulation of the present disclosure includes a nanoparticle. Nanoparticles suitable for use in the inventive formulations are known by those of ordinary skill in the art. For example, a formulation of the present disclosure can include an effective amount of at least one of the modified siRNA compositions and gold nanoparticles, iron-core magnetic enrichable nanoparticles, chitosan nanoparticles or combinations thereof.

In one embodiment, a formulation of the present disclosure can include an effective amount of at least one of the modified siRNA compositions and a transfection agent such as polyethylenimine, polyethylenimine hydrochloride, a deacylated polyethylenimine and oligofectamine However, other transfections agents for use in the inventive formulations are known by those of ordinary skill in the art.

In some instances, the short-interfering nucleic acid compositions of the present disclosure may be formulated into compositions and dosage forms according to methods known in the art. In certain embodiments, the formulated compositions may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

In some embodiments, the formulations of the present disclosure include a solid pharmaceutical agent that is compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in a subject's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Therefore, in certain embodiments, glidants can be added to formulations to improve the flowability of a non-compacted solid agent and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

A formulated pharmaceutical composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried and then screened and/or milled to the desired particle size. The granulate may then be tableted, or other excipients may be added prior to tableting, such as a glidant and/or a lubricant. A tableting composition may be prepared conventionally by dry blending. For example, the blended composition of the actives and excipients may be compacted into a slug or a sheet and then comminuted into compacted granules. The compacted granules may subsequently be compressed into a tablet.

In other embodiments, as an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting. A capsule filling may include any of the aforementioned blends and granulates that were described with reference to tableting, however, they are not subjected to a final tableting step.

In liquid pharmaceutical compositions (i.e., formulations) of the present disclosure, the agent (modified siRNA composition) and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. The liquid formulation may be used as an injectable, enteric, or emollient type of formulation. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

In some embodiments, liquid pharmaceutical compositions of the present disclosure may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

In other embodiments, the liquid composition of the present disclosure may also contain a buffer, such as gluconic acid, lactic acid, citric acid or acetic acid, sodium gluconate, sodium lactate, sodium citrate, or sodium acetate.

Preservatives and chelating agents, such as alcohol, sodium benzoate, butylated hydroxy toluene, butylated hydroxyanisole and ethylenediamine tetraacetic acid, may be added at levels safe for ingestion to improve storage stability. Solid and liquid compositions may also be dyed using any pharmaceutically acceptable colorant to improve their appearance and/or facilitate patient identification of the product and unit dosage level.

A dosage formulation of the present disclosure may be a capsule containing the composition, for example, a powdered or granulated solid composition of the disclosure, within either a hard or soft shell. The shell may be made from gelatin and optionally contain a plasticizer such as glycerin and sorbitol, and an opacifying agent or colorant.

In certain instances, a formulation of the present disclosure will include an effective amount of at least one of the modified siRNA compositions without a targeting agent, carrier or other vehicle for targeting a cancer cell. In one embodiment, such a formulation will be liquid, and suitable for injection. In some instances, the liquid composition will be formulated for intravenous injection to a subject. In other instances, the liquid composition will be formulated for injection directly into a tumor or a cell thereof.

### Methods for Treating Cancer

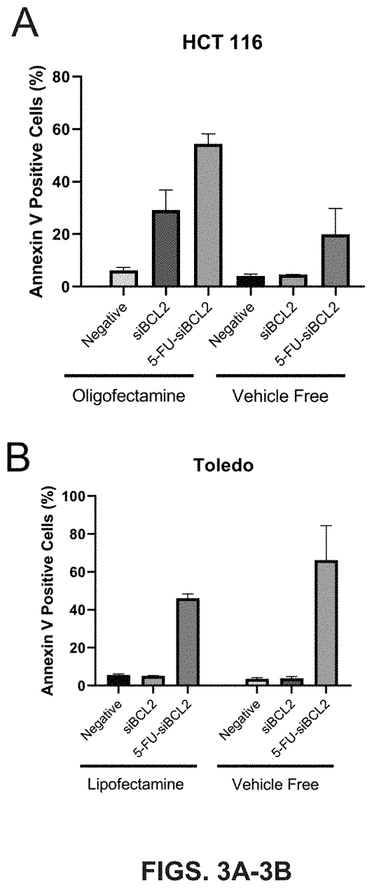

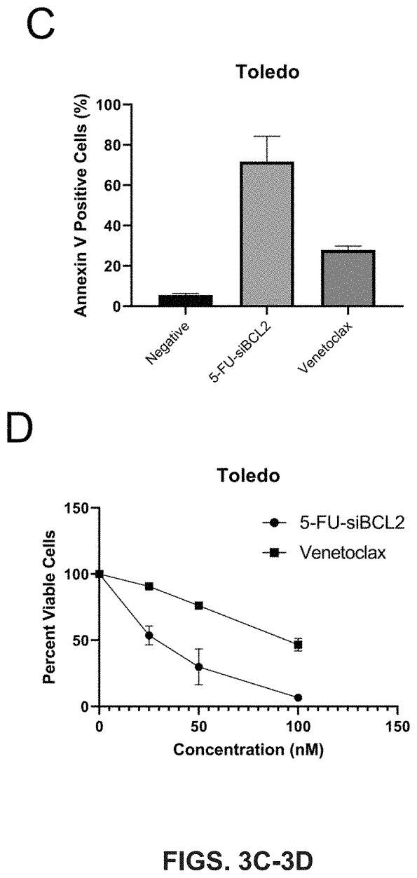

As stated above, the modified short-interfering ribosomal nucleic acid compositions of the present disclosure and formulations thereof show unexpected and exceptional anti-cancer activity when compared to that exhibited by exogenous expression of a corresponding unmodified siRNA and/or other known cancer therapies. See FIGS. 3A-3C. Therefore, another aspect of the present disclosure provides a method for treating cancer in a mammal by administering to the mammal an effective amount of one or more of the modified siRNA compositions of the present disclosure, or formulations thereof.

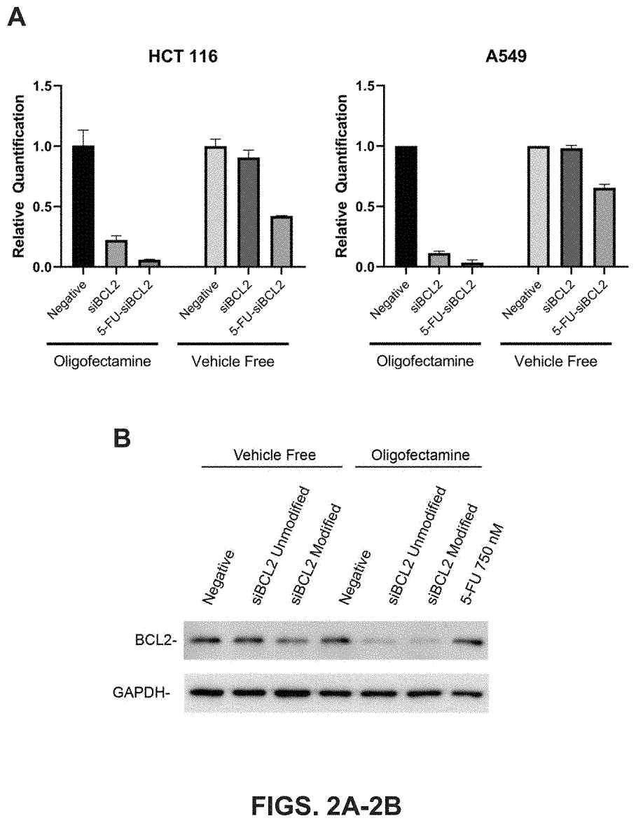

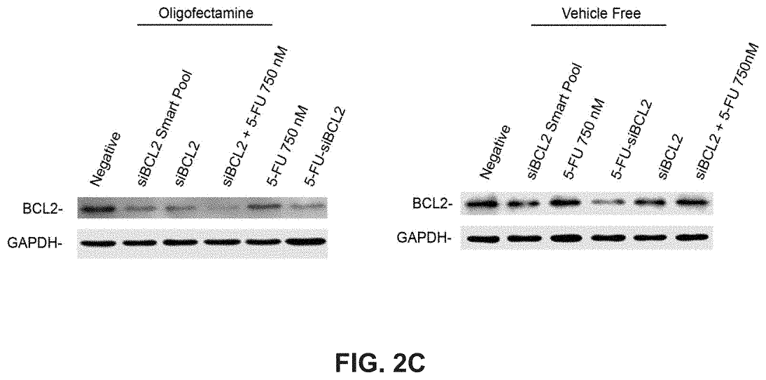

As shown in FIGS. 2A through 2C, exemplary modified siRNA compositions (i.e., SEQ ID NO: 2) of the present disclosure bind to BCL-2 mRNA and suppress BCL-2 protein expression in cancer cells with or without the presence of a delivery vehicle.

In addition and as shown in FIGS. 3A-3B, the exemplary modified siRNA's described herein reduce colorectal cancer and lymphoma by inducing apoptosis as well as cell viability. More specifically, modified siRNAs having all U bases replaced with a 5-FU molecule, as set forth in SEQ ID NO: 2 reduce colorectal cancer cell viability (FIG. 3A) and lymphoma cell viability (FIG. 3B). Moreover, the present modified siRNA compositions were tested and found to provide an unexpected increase in therapeutic efficacy in lymphoma cells when compared to known lymphoma cancer therapeutic compositions (e.g., Venetoclax). See, for example, FIGS. 3C and 3D. Therefore, the disclosed methods for treating cancer include administering one or more modified short-interfering ribosomal nucleic acid compositions of the present disclosure to a subject with cancer.

In certain embodiments, the modified short-interfering ribosomal nucleic acid composition can be administered as a formulation that includes a modified nucleic acid composition as described above. In specific embodiments, the nucleic acid compositions of the present disclosure can be administered in the absence of a delivery vehicle or pharmaceutical carrier (i.e., naked). See, for example, FIGS. 2A and 2C.

The term “subject” as used herein refers to any mammal. The mammal can be any mammal, although the methods herein are more typically directed to humans. The phrase “subject in need thereof” as used herein is included within the term subject and refers to any mammalian subject in need of a treatment, particularly cancer or has a medically determined elevated risk of a cancerous or pre-cancerous condition. In specific embodiments, the subject includes a human cancer patient.

The terms “treatment”, “treat” and “treating” are synonymous with the term “to administer an effective amount”. These terms shall mean the medical management of a subject with the intent to cure, ameliorate, stabilize, reduce one or more symptoms of or prevent a disease, pathological condition, or disorder such as cancer. These terms, are used interchangeably and include the active treatment, that is, treatment directed specifically toward the improvement of a disease, pathological condition, or disorder, and also include causal treatment, that is, treatment directed toward removal of the cause of the associated disease, pathological condition, or disorder. In addition, treating includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. In a specific embodiment, treatment of a disease, such as a cancer includes inhibiting proliferation of cancer cells. In some embodiments, the treatment of a cancer can be determined by detecting a reduction in the amount of proliferating cancer cells in a subject, a reduction in tumor growth or tumor size.

In certain embodiments, the modified short-interfering ribosomal nucleic acid compositions of the present disclosure are used to treat cancer.

The term “cancer”, as used herein, includes any disease caused by uncontrolled division and growth of abnormal cells, including, for example, the malignant and metastatic growth of tumors. The term “cancer” also includes pre-cancerous conditions or conditions characterized by an elevated risk of a cancerous or pre-cancerous condition. The cancer or pre-cancer (neoplastic condition) can be located in any part of the body, including the internal organs and skin. As is well known, cancer spreads through a subject by invading the normal, non-cancerous tissue surrounding the tumor, via the lymph nodes and vessels, and by blood after the tumor invades the veins, capillaries and arteries of a subject. When cancer cells break away from the primary tumor (“metastasize”), secondary tumors arise throughout an afflicted subject forming metastatic lesions.

Some non-limiting examples of applicable cancer cells for treatment using the present methods include the lungs, colon, rectum, blood, lymphatic system or immune system. The cancer or neoplasm can also include the presence of one or more carcinomas, sarcomas, lymphomas, blastomas, or teratomas (germ cell tumors).

In specific examples, modified siRNA compositions have been shown to reduce cancer cell proliferation by increasing apoptosis across the following experimental models, colorectal cancer cells (FIG. 3A), and lymphoma cells (FIGS. 3B-3D).

In some embodiments, the subject administered treatment including a modified siRNA of the present disclosure has colorectal cancer, or has a medically determined elevated risk of getting colorectal cancer.

In certain embodiments, a subject of the present disclosure has lung cancer, or has a medically determined elevated risk of getting lung cancer.

In other embodiments, the subject has lymphoma, or has a medically determined elevated risk of getting lymphoma.

According to the present disclosure, methods of treating cancer include administration of one or more short-interfering ribosomal nucleic acid compositions of the present by any of the routes commonly known in the art. This includes, for example, (1) oral administration; (2) parenteral administration, for example, by subcutaneous, intramuscular or intravenous injection; (3) topical administration; or (4) intravaginal or intrarectal administration; (5) sublingual or buccal administration; (6) ocular administration; (7) transdermal administration; (8) nasal administration; and (9) administration directly to the organ or cells in need thereof.

In specific embodiments, the modified siRNA compositions of the present disclosure are administered to a subject by injection. In one embodiment, a therapeutically effective amount of a modified siRNA composition is injected intravenously. In another embodiment, a therapeutically effective amount of a modified siRNA composition is injected intraperitoneally or subcutaneously to a tumor or cell thereof.

The amount (dosage) of nucleic acid compositions of the present disclosure being administered depends on several factors, including the type and stage of the cancer, presence or absence of an auxiliary or adjuvant drug, and the subject's weight, age, health, and tolerance for the agent. Depending on these various factors, the dosage may be, for example, about 2 mg/kg of body weight, about 5 mg/kg of body weight, about 10 mg/kg of body weight, about 15 mg/kg of body weight, about 20 mg/kg of body weight, about 25 mg/kg of body weight, about 30 mg/kg of body weight, about 40 mg/kg of body weight, about 50 mg/kg of body weight, about 60 mg/kg of body weight, about 70 mg/kg of body weight, about 80 mg/kg of body weight, about 90 mg/kg of body weight, about 100 mg/kg of body weight, about 125 mg/kg of body weight, about 150 mg/kg of body weight, about 175 mg/kg of body weight, about 200 mg/kg of body weight, about 250 mg/kg of body weight, about 300 mg/kg of body weight, about 350 mg/kg of body weight, about 400 mg/kg of body weight, about 500 mg/kg of body weight, about 600 mg/kg of body weight, about 700 mg/kg of body weight, about 800 mg/kg of body weight, about 900 mg/kg of body weight, or about 1000 mg/kg of body weight, wherein the term “about” is generally understood to be within +10%, 5%, 2%, or 1% of the indicated value. The dosage may also be within a range bounded by any two of the foregoing values. Routine experimentation may be used to determine the appropriate dosage regimen for each individual subject by monitoring the composition or formulation thereofs effect on the cancerous or pre-cancerous condition, or the effect of the modified siRNA's expression on BCL-2 protein or nucleic acid sequence expression, or the disease pathology, all of which can be frequently and easily monitored according to methods known in the art. Depending on the various factors discussed above, any of the above exemplary doses of nucleic acid can be administered once, twice, or multiple times per day, week or month.

The ability of the nucleic acid compositions described herein, and optionally, any additional chemotherapeutic agent for use with the current methods can be determined using pharmacological models well known in the art, such as cytotoxic assays, apoptosis staining assays, xenograft assays, and binding assays.

The inventive short-interfering nucleic acid compositions described herein may or may not also be co-administered with one or more chemotherapeutic agents, which may be auxiliary or adjuvant drugs different from a nucleic composition described herein.

As used herein, “chemotherapy” or the phrase a “chemotherapeutic agent” is an agent useful in the treatment of cancer. Chemotherapeutic agents useful in conjunction with the methods described herein include, for example, any agent that modulates BMII, either directly or indirectly. Examples of chemotherapeutic agents include: anti-metabolites such as methotrexate and fluoropyrimidine-based pyrimidine antagonist, 5-fluorouracil (5-FU) (Carac® cream, Efudex®, Fluoroplex®, Adrucil®) and S-1; antifolates, including polyglutamatable antifolate compounds; raltitrexed (Tomudex®), GW1843 and pemetrexed (Alimta®) and non-polyglutamatable antifolate compounds; nolatrexed (Thymitaq®), plevitrexed, BGC945; folic acid analogs such as denopterin, methotrexate, pteropterin, trimetrexate; and purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine. In a specific embodiment of the current disclosure, the chemotherapeutic agent is a compound capable of inhibiting the expression or activity of genes, or gene products involved in signaling pathways implicated in aberrant cell proliferation or apoptosis, such as, for example, BCL2, thymidylate synthase or E2F3; and pharmaceutically acceptable salts, acids or derivatives of any of the above.

E2F transcription factor 3, E2F3 (RefSeq NG_029591.1, NM_001243076.2, NP_001230005.1) is a transcription factor that binds DNA and interacts with effector proteins, including but not limited to, retinoblastoma protein to regulate the expression of genes involved in cell cycle regulation. Therefore, any drug that inhibits the expression of E2F3 may be considered herein as a co-drug.

Thymidylate synthase (RefSeq: NG_028255.1, NM_001071.2, NP_001062.1) is a ubiquitous enzyme, which catalyses the essential methylation of dUMP to generate dTMP, one of the four bases which make up DNA. The reaction requires CH H₄-folate as a cofactor, both as a methyl group donor, and uniquely, as a reductant. The constant requirement for CH H₄-folate means that thymidylate synthase activity is strongly linked to the activity of the two enzymes responsible for replenishing the cellular folate pool: dihydrofolate reductase and serine transhydroxymethylase. Thymidylate synthase is a homodimer of 30-35 kDa subunits. The active site binds both the folate cofactor and the dUMP substrate simultaneously, with the dUMP covalently bonded to the enzyme via a nucleophilic cysteine residue (See, Carreras et al, Annu. Rev. Biochem., (1995) 64:721-762). The thymidylate synthase reaction is a crucial part of the pyrimidine biosynthesis pathway which generates dCTP and dTTP for incorporation into DNA. This reaction is required for DNA replication and cell growth. Thymidylate synthase activity is therefore required by all rapidly dividing cells such as cancer cells. Due to its association with DNA synthesis, and therefore, cellular replication, thymidylate synthase has been the target for anti-cancer drugs for many years. Non-limiting examples of thymidylate synthase inhibitors include folate and dUMP analogs, such as 5-fluorouracil (5-FU). Any drug that inhibits the expression of thymidylate synthase may be considered herein as a co-drug.

B-cell lymphoma 2 (BCL2), (RefSeq NG_009361.1, NM_000633, NP_000624) including isoform α (NM_000633.2, NP_000624.2) and β (NM_000657.2, NP_000648.2) thereof, are encoded by the Bcl-2 gene, which is a member of the BCL2 family of regulator proteins that regulate mitochondria regulated cell death via the intrinsic apoptosis pathway. BCL2 is an integral outer mitochondrial membrane protein that blocks the apoptotic death of cell cells by binding BAD and BAK proteins.

In specific embodiments, the modified siRNA compositions of the present disclosure are administered in conjunction with a known BCL-2 inhibitors such as for example, Venetoclax (C₄₅H₅₀ClN₇O₇S, Genentech, Inc.), antisense oligonucleotides, such as Oblimersen (Genasense; Genta Inc.,), BH3 mimetic small molecule inhibitors including, ABT-737 (Abbott Laboratories, Inc.), ABT-199 (Abbott Laboratories, Inc.), and Obatoclax (Cephalon Inc.). In one embodiment, the modified siRNA composition of the present disclosure is administered to a subject with Ventoclax. In a specific embodiment, the modified siRNA composition of the present disclosure is administered with Ventoclax to a subject with lymphoma.

The chemotherapeutic agent may be administered before, during, or after commencing therapy with the nucleic acid composition.

In a specific embodiment, the other nucleic acid is a short hairpin RNA (shRNA), miRNA, modified miRNA, or other form of nucleic acid that binds to or is complementary to a portion of a BCL-2 nucleic acid sequence.

If desired, the administration of a short-interfering nucleic acid composition described herein may be combined with one or more non-drug therapies, such as, for example, radiotherapy, and/or surgery. As well known in the art, radiation therapy and/or administration of the chemotherapeutic agent (in this case, the nucleic acid composition described herein, and optionally, any additional chemotherapeutic agent) may be given before surgery to, for example, shrink a tumor or stop the spread of the cancer before the surgery. As also well known in the art, radiation therapy and/or administration of the chemotherapeutic agent may be given after surgery to destroy any remaining cancer.

Examples have been set forth below for the purpose of illustration and to describe certain specific embodiments of the invention. However, the scope of this invention is not to be in any way limited by the examples set forth herein.

### EXAMPLES

### Example 1. Materials and Methods

Modified short-interfering RNAs. All siRNAs were synthesized as single strands by an automated oligonucleotide synthesis process and purified by HPLC. The two strands (sense and antisense) were annealed to make the double-stranded modified siRNAs that bind to BCL-2 mRNA. For modified siRNA that binds to BCL-2 mRNA containing a 5 fluorouracil, a process referred to as “2′-ACE RNA synthesis” was used. The 2′-ACE RNA synthesis is based on a protecting group scheme in which a silylether is employed to protect the 5′-hydroxyl group in combination with an acid-labile orthoester protecting group on the 2′-hydroxy (2′-ACE). This combination of protecting groups is then used with standard phosphoramidite solid-phase synthesis technology. See, for example, S. A. Scaringe, F. E. Wincott, and M. H. Caruthers, J. Am. Chem. Soc., 120 (45), 11820-11821 (1998); International PCT Application WO/1996/041809; M. D. Matteucci, M. H. Caruthers, J. Am. Chem. Soc., 103, 3185-3191 (1981); S. L. Beaucage, M. H. Caruthers, Tetrahedron Lett. 22, 1859-1862 (1981), the entire contents of each of which are expressly incorporated herein. Some exemplary structures of the protected and functionalized ribonucleoside phosphoramidites currently in use are shown below.

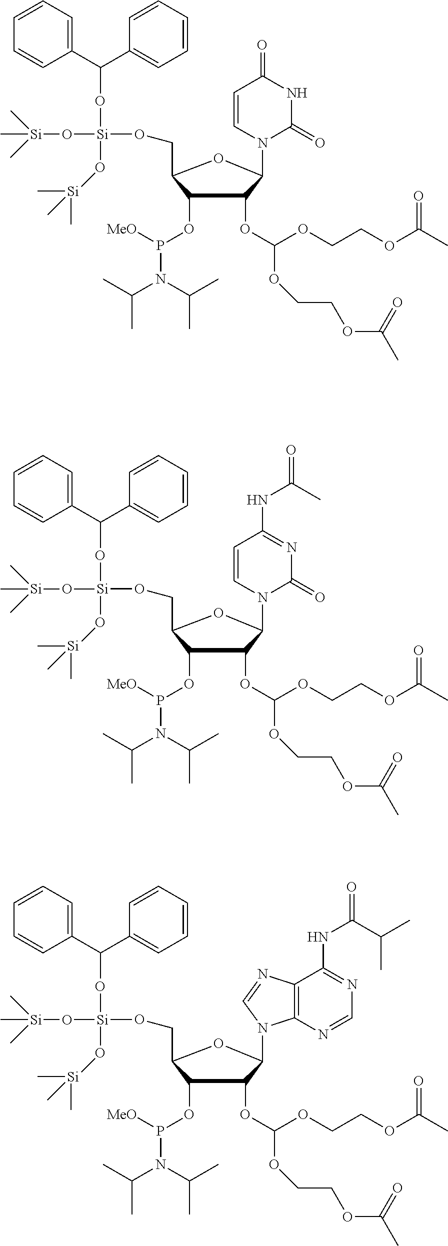

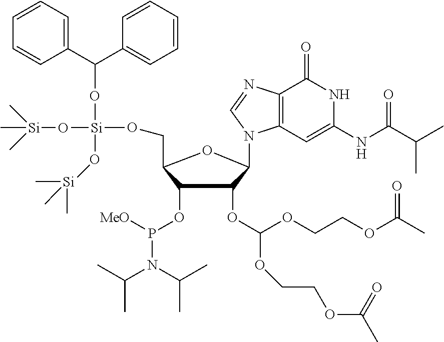

Cell culture. The human colon cancer cell line, HCT116, human lymphoma cell line, Toledo, and the human lung cancer cell line, A459, were obtained from the American Type Culture Collection (ATCC) and maintained in various types of media. Specifically, HCT116, was cultured in McCoy's 5A media (Thermo Fischer), Toledo was maintained in RPMI media (Thermo Fischer) and A459 cells were cultured in F12K media (Thermo Fischer). Each media was supplemented with 10% fetal bovine serum (Thermo Fischer).

qRT-PCR analysis. Twenty-four hours prior to transfection 1×10⁵ cells were plated in 6-well plates. Cells were either transfected using Oligofectamine (Thermo Fischer) or no transfection vehicle, with 50 nM control (scrambled) siRNA, unmodified siBCL2 or modified siBCL2 siRNA. Twenty-four hours later, RNA was isolated using Trizol (Thermo Fischer). cDNA was synthesized using the High Capacity cDNA Synthesis Kit (Thermo Fischer). Real-time qRT-PCR was carried out using siBCl2 and GAPDH specific TaqMan primers (Thermo Fischer). Expression level of BCL-2 was calculated using the ΔΔCT method based on the internal control GAPDH, normalized to the control group and plotted as relative quantification.

Western immunoblot analysis. Twenty-four hours prior to transfection 1×10⁵ cells were plated in 6 well plates. Cells were either transfected using Oligofectamine (Thermo Fischer) or no transfection vehicle, with 50 nM control (scrambled) siRNA (Thermo Fischer), unmodified siBCL2 or an exemplary modified siBCL2 siRNA. Three days following transfection protein was collected in RIPA buffer with protease inhibitor (Sigma). Equal amounts of protein (15 μg), were separated on 12% sodium dodecyl sulfate-polyacrylamide gels as described in Laemmli U K. Nature. 1970; 227(5259) pp. 680-685, the entire contents of which is incorporated herein by reference. Proteins were probed with anti-BCL2 antibody (1:1000) (Thermo Fischer) and anti-GAPDH antibody (1:100000) (Santa Cruz). Horseradish peroxidase conjugated secondary antibodies against mouse or rabbit (1:5000, Santa Cruz Biotech Inc.) were added. Protein bands were then visualized with autoradiography film using SuperSignal West Pico Chemiluminescent Substrate (Thermo Fischer).

Apoptosis and cell viability assay. To measure apoptosis induced by exemplary modified BCL2 binding siRNA compositions as well as cell viability following treatment with exemplary modified siRNAs or Venetoclax, a fluorescein isothiocyanate (FITC)-Annexin assay was used (Becton Dickinson). Twenty-four hours before transfection, cells were plated into 6 well plates (1×105) cells per well. Cells were transfected with various concentrations of exemplary modified siRNAs or treated with various concentrations of Venetoclax. Forty-eight hours after transfection, cells were harvested, stained with propidium iodide and anti-annexin-V antibody (Annexin V-FITC Apoptosis Detection kit, Invitrogen, CA, USA) following the manufacturer's protocol, and stained cells were detected by flow cytometry.

Statistical analysis. All statistical analyses were performed with GraphPad Prism 8 software. The statistical significance between two groups was determined using Student's t-test. Foe comparison of more than two groups, one way ANOVA was used. Data is expressed as mean±standard deviation (S.D.)

### Example 2: Modified siRNA Nucleic Acids have Anti-Cancer Activity

In the following experiments, all uracil bases in the sense and antisense strand of an anti-BCL-2 siRNA molecule (SEQ ID NO: 1) were replaced by 5-FU to form the exemplary modified siRNA set forth in SEQ ID NO: 2. See FIG. 1B. In order to test if this siRNA retains the ability to inhibit BCL-2 and be delivered into cancer cells with no transfection vehicle, HCT116 colon cancer cells and A549 lung cancer cells were transfected with 50 nM control siRNA, unmodified siRNA that binds to a portion of BCL2 or a modified siBCL2 of SEQ ID NO. 2 with or without transfection vehicle. See FIG. 2A. qRT-PCR was used to assess the expression of BCL-2 after transfection.

The data depicted in FIG. 2A shows that with transfection vehicle, both unmodified siBCL2 and modified siBCL2 reduced the expression of BCL-2 in cells, while in the absence of transfection vehicle, unmodified siBCL2 had no effect on the level of BCL-2 mRNA, while modified siBCL2 decreased the level of BCL2 mRNA.

The effects of the exemplary modified siRNA compositions on BCL-2 protein levels were also examined. As shown in FIGS. 2B and 2C, a similar effect was shown at the protein level when compared to mRNA levels. With transfection vehicle, both unmodified siBCL2 and modified siBCL2 inhibited BCL-2 protein expression. Without transfection vehicle, only modified siBCL2 was able to inhibit BCL-2 expression. Cancer cells were also treated cells with 5-FU alone to show that 5-FU alone does not result in reduction of BCL-2 expression. See FIGS. 2C and 2D. These data suggest that replacement of the uracil bases of an siRNA that binds to a portion of BCL-2 does not disrupt target binding and also allows the siRNA to enter the cell with no transfection vehicle, which is not shown by 5-FU alone.

5-FU-siBCL2 triggers apoptosis and is more effective than Venetoclax. In order to measure the therapeutic effects of 5-FU-siBCL2, and compare it to known cancer therapeutics (i.e., Venetoclax), apoptosis assays and flow cytometry were used to assess the induction of apoptosis as well as cell viability following treatment with exemplary modified siRNA molecules of the present disclosure, siBCL2 or Venetoclax.

FIGS. 3A-3B show that colon cancer cells (HCT116, FIG. 3A) as well as lymphoma cells (Toledo, FIG. 3B) are killed more effectively by administration of the exemplary modified siRNA set forth in SEQ ID NO: 2 than unmodified siBCL2 control siRNA.

The therapeutic efficacy of the exemplary modified siRNA set forth in SEQ ID NO: 2 was also compared to that of FDA approved BCL-2 selective inhibitor, Venetoclax (ABT-199) in lymphoma cells. See FIGS. 3C-3D. FIG. 3C reveals that the exemplary modified siRNA set forth in SEQ ID NO: 2 was more effective at inducing apoptosis than Venetoclax. In addition, it was observed that the exemplary modified siRNA set forth in SEQ ID NO: 2 was effective at inhibiting cell viability at a lower dose than Venetoclax. See FIG. 3D.

In summary, the present disclosure shows that novel siRNA compositions can be used to effectively treat colorectal, lung or lymphomas without the aid of a delivery vehicle and without interfering with target binding and interaction. Furthermore, the exemplary modified siRNA set forth in SEQ ID NO: 2 more effective than known BCL-2 inhibitors (e.g., Venetoclax) in inducing apoptosis and inhibiting lymphoma cell viability.

1. A short-interfering ribosomal nucleic acid composition comprising a modified nucleotide sequence that comprises at least one uracil nucleic acid replaced by a 5-fluorouracil (5-FU) molecule, wherein the short-interfering ribosomal nucleic acid (siRNA) binds to the BCL-2 mRNA sequence set forth in SEQ ID NO: 1.

2. The short-interfering nucleic acid composition of claim 1, wherein at least two of the uracil nucleic acids in the modified nucleotide sequence are each replaced by a 5-FU molecule.

3. The short-interfering nucleic acid composition of claim 1, wherein all of the uracil nucleic acids in the modified nucleotide sequence are replaced by a 5-FU molecule.

4. The short-interfering nucleic acid composition of claim 1, wherein said modified nucleotide sequence comprises a first strand and a second strand of nucleic acids.

5. The short-interfering nucleic acid composition of claim 4, wherein said first strand and said second strand are complementary to one another.

6. The short-interfering nucleic acid composition of claim 4, wherein at least one uracil nucleic acid in said first strand is replaced by a 5-FU molecule.

7. The short-interfering nucleic acid composition of claim 6, wherein none of the uracil nucleic acids in said second strand are replaced by a 5-FU molecule.

8. The short-interfering nucleic acid composition of claim 7 comprising the modified nucleotide sequence set forth in SEQ ID NO: 3 or SEQ ID NO: 4.

9. The short-interfering nucleic acid composition of claim 6, wherein at least one uracil nucleic acid in said second strand is replaced by a 5-FU molecule.

10. The short-interfering nucleic acid composition of claim 9, wherein all of the uracil nucleic acids in said first strand and all of the uracil nucleic acids in said second strand are replaced by a 5-FU molecule.

11. The short-interfering nucleic acid composition of claim 10 comprising the modified nucleotide sequence set forth in SEQ ID NO: 2.

12. A pharmaceutical composition comprising a short-interfering nucleic acid composition of claim 1.

13. The pharmaceutical composition of claim 12, wherein the short-interfering nucleic acid composition comprises a modified nucleotide sequence selected from the group consisting of SEQ ID NO: 2, SEQ ID NO: 3 and SEQ ID NO: 4.

14. The pharmaceutical composition of claim 14, wherein the short-interfering nucleic acid composition comprises the modified nucleotide sequence set forth in SEQ ID NO: 2.

15. A method for treating cancer comprising administering to a subject an effective amount of a short-interfering nucleic acid composition of claim 1, wherein said subject has cancer, and wherein progression of said cancer is inhibited.

16. The method of claim 15, wherein said subject is a human.

17. The method of claim 16, wherein said subject has a cancer selected from the group consisting of lung cancer, colorectal cancer or lymphoma.

18. The method of claim 17, wherein said subject has lymphoma.

19. The method of claim 15, further comprising administering to the subject a chemotherapeutic agent.


SEQUENCE LISTING (4 sequences)

SEQ ID NO: 1 - RNA, 23 nt, Artificial Sequence
  sense strand of double stranded short-interfering RNA against BCL-2 mRNA sequence
ggaugccuuuguggaacuguauu

SEQ ID NO: 2 - RNA, 23 nt, Artificial Sequence
  sense strand of double stranded short-interfering RNA against BCL-2 mRNA sequence with all with all uracils modified in both strands
  misc_feature (4)..(4): fluorouracil
  misc_feature (8)..(10): fluorouracil
  misc_feature (12)..(12): fluorouracil
  misc_feature (18)..(18): fluorouracil
  misc_feature (20)..(20): fluorouracil
  misc_feature (22)..(23): fluorouracil
ggaugccuuuguggaacuguauu

SEQ ID NO: 3 - RNA, 23 nt, Artificial Sequence
  sense strand of double stranded short-interfering RNA against BCL-2 mRNA sequence with all with all uracils modified in sense strand and no uracils modified in complementary strand
  misc_feature (4)..(4): fluorouracil
  misc_feature (8)..(10): fluorouracil
  misc_feature (12)..(12): fluorouracil
  misc_feature (18)..(18): fluorouracil
  misc_feature (20)..(20): fluorouracil
  misc_feature (22)..(23): fluorouracil
ggaugccuuuguggaacuguauu

SEQ ID NO: 4 - RNA, 23 nt, Artificial Sequence
  antisense strand of double stranded short-interfering RNA against BCL-2 mRNA sequence with all uracils modified and no uracils modified in complementary strand
  misc_feature (1)..(2): fluorouracil
  misc_feature (5)..(5): fluorouracil
  misc_feature (17)..(18): fluorouracil
  misc_feature (23)..(23): fluorouracil
uuccuacggaaacaccuugacau